(19) United States
(12) Patent Application Publication
(10) Pub. No.: US 2020/0289595 A1
(43) Pub. Date: Sep. 17, 2020

(54) POWDERED CANNABIS EXTRACT

(71) Applicants: CannTrust Inc., Vaughan (CA); Single Dose Solutions Inc., Toronto (CA)

(72) Inventors: Ilana Deborah PLATT, Toronto (CA); Claudio GEMMITI, Toronto (CA)

(73) Assignees: CannTrust Inc., Vaughan, ON (CA); Single Dose Solutions Inc., Toronto, ON (CA)

(21) Appl. No.: 16/636,225

(22) PCT Filed: Aug. 2, 2018
(86) PCT No.: PCT/CA2018/050945

§ 371 Date: Feb. 3, 2020

Related U.S. Application Data
(60) Provisional application No. 62/540,777, filed on Aug. 3, 2017.

(51) Int. Cl. A61K 36/185; A61K 9/50; A61K 9/00; A23L 3/44; A23L 2/56; A23L 2/395; A23L 33/105; A23L 33/19; A23L 29/30; A23P 10/30; A23P 10/40; B65B 29/02; B65D 85/804

(52) U.S. Cl. CPC A61K 36/185; A23V 2002/00; A61K 9/0095; A23L 3/44; A23L 2/56; A23L 2/395; A23L 33/105; A23L 33/19; A23L 29/35; A23P 10/30; A23P 10/40; B65B 29/022; B65D 85/8043; A61K 2236/37; A61K 9/5015

(57) ABSTRACT

Powdered cannabis extracts and methods for obtaining powdered cannabis extracts are described. In an aspect, the present application describes a method of preparing a single-serve container. The single-serve container may be configured for receipt in a single-serve brewing machine. The method may include: obtaining cannabis extract; processing the cannabis extract to obtain powdered cannabis extract and adding the powdered cannabis extract to the single-serve container; adding a flavoring agent to the single serve container; and sealing the single-serve container.

### TECHNICAL FIELD

The present application relates to cannabis and, more particularly, to cannabis products such as single-serve beverage pods containing powdered cannabis extract such as freeze-dried microencapsulated cannabis extract and methods of preparing cannabis products, such as powdered cannabis extracts.

### BACKGROUND

Cannabis, which is commonly known as marijuana, is often used as a medicine for the treatment of a variety of conditions. Cannabis contains numerous cannabinoids, such as delta-9-tetrahydrocannabinolic Acid (THCA). Acidic cannabinoids, such as THCA and cannabidiolic acid (CBDA) may be converted to more active cannabinoids, through a process known as decarboxylation. For example, THCA is converted to delta-9-tetrahydrocannabinol (THC) through decarboxylation.

Decarboxylation is typically performed by smoking cannabis. The heat generated during smoking decarboxylates the inactive cannabinoids, such as THCA, into the active form, such as THC.

The use of smoking as a means for delivering the active ingredients in cannabis to a patient has a number of problems. For example, ensuring a proper dosage for medical marijuana users is difficult with smoking since each patient has different smoking tendencies which will affect the dose. More particularly, medical marijuana is often prescribed as a dose per day by weight for a patient. An example of a medical marijuana prescription may be 0.5 g of marijuana taken two times per day for a period of 30 days. Different users may, however, inhale a different amount of the active ingredients when smoking. For example, the actual dose for a patient (i.e. the amount actually consumed) will depend on variables such as the elapsed time between inhales, the amount of time that the patient holds the smoke in, the tightness of the cannabis cigarette, the moisture content of the cigarette (which may affect the burn rate between breaths) and other variables.

Also, some patients may be reluctant to smoke marijuana because of a stigma associated with smoking marijuana, perceived health concerns associated with smoking, or their inability to smoke effectively due to other medical conditions.

Thus, there is a need for improved methods of delivering medical marijuana.

### BRIEF DESCRIPTION OF THE DRAWINGS

Reference will now be made, by way of example, to the accompanying drawings which show embodiments of the present application, and in which:

FIG. 1 is a top perspective view of a soft-shell single-serve container according to an embodiment of the present disclosure;

FIG. 2 is a bottom perspective view of the soft-shell single-serve container of FIG. 1;

FIG. 3 is a cross-sectional view of the single-serve container of FIG. 1;

FIG. 4 is a perspective view of an example hard-shell single-serve container according to an embodiment of the present disclosure;

FIG. 5 is a cross-sectional view of the single-serve container of FIG. 4; and

FIG. 6 is a flowchart of a method for facilitating rapid extraction of cannabis into a liquid.

Like reference numerals are used in the drawings to denote like elements and features.

### DETAILED DESCRIPTION OF EXAMPLE EMBODIMENTS

In an aspect, the present application may describe a method of preparing a single-serve container. The single-serve container may be configured for receipt in a single-serve brewing machine. The method may include: obtaining cannabis extract; processing the cannabis extract to obtain powdered cannabis extract and adding the powdered cannabis extract to the single-serve container; adding a flavoring agent to the single serve container; and sealing the single-serve container.

In some implementations, obtaining the cannabis extract may include decarboxylating the cannabis. In some implementations, decarboxylating the cannabis may include heating the cannabis in an oven. In some implementations, heating may include baking at a temperature of between 120 and 140 degrees Celsius for a time period in the range of 30 to 90 minutes.

In some implementations, the flavoring agent may be one of tea, coffee or chocolate, latte or cappuccino.

In some implementations, processing the cannabis extract to obtain powdered cannabis extract may include microencapsulating the cannabis extract by freeze drying.

In some implementations, the cannabis extract may be microencapsulated in sodium caseinate.

In some implementations, processing the cannabis extract may include mixing the cannabis extract with an absorbent carrier.

In some implementations, processing the cannabis extract may include mixing sodium caseinate together with water and the cannabis extract to obtain a mixture; homogenizing the mixture; and freeze drying the homogenized mixture.

In some implementations, the mixture includes approximately 10% sodium caseinate by weight, approximately 10% cannabis extract by weight and approximately 80% water by weight.

In some implementations, mixing further includes mixing maltodextrin with the sodium caseinate, water and cannabis extract.

In some implementations, the mixture includes approximately 10% sodium caseinate by weight, approximately 10% cannabis extract by weight, approximately 10% maltodextrin by weight and approximately 70% water by weight.

In an aspect, a single-serve container is described. The single-serve container is produced according to the method described herein.

In yet another aspect, a method of converting liquid cannabis extract into a solid may be described. The method includes: obtaining the liquid cannabis extract; and processing the cannabis extract to obtain powdered cannabis extract.

In some implementations, obtaining the cannabis extract may include decarboxylating cannabis.

In some implementations, decarboxylating the cannabis may include heating the cannabis in an oven.

In some implementations, heating may include baking at a temperature of between 120 and 140 degrees Celsius for a time period in the range of 30 to 90 minutes.

In some implementations, processing the cannabis extract to obtain powdered cannabis extract may include microencapsulating the cannabis extract by freeze drying.

In some implementations, the cannabis extract may be microencapsulated in sodium caseinate.

In some implementations, processing the cannabis extract may include: mixing sodium caseinate together with water and the cannabis extract to obtain a mixture; homogenizing the mixture; and freeze drying the homogenized mixture.

In some implementations, the mixture may include approximately 10% sodium caseinate by weight, approximately 10% cannabis extract by weight and approximately 80% water by weight.

In some implementations, mixing may further include mixing maltodextrin with the sodium caseinate, water and cannabis extract.

In some implementations, the mixture may further include approximately 10% sodium caseinate by weight, approximately 10% cannabis extract by weight, approximately 10% maltodextrin by weight and approximately 70% water by weight.

As will be described in greater detail below, in some embodiments, a method is described for delivering THC and other active components of marijuana using existing single-serve beverage machines, such as single-serve brewing machines. Single-serve brewing machines are machines that receive single-serve containers having specific dimensions, shapes or features. The single-serve containers are often called pods. The brewing machines may, for example, be Keurig™ brewing machines (or Keurig™ compatible brewing machines) which are configured to receive a K-Cup™ or a K-cup compatible pod. By way of further example, the brewing machines may be Tassimo™ brewing machines and may be configured to receive a T Disc™ or T-disc compatible pods. Other types of brewing machines are also contemplated. By way of further example, in some embodiments the brewing machines may be Nespresso™ brewing machines which are configured to receive pods compatible with such machines.

The brewer (which is also referred to as a brewing machine herein) may be configured to pierce the pod with one or more spray nozzle(s) which passes water into the pod. More specifically, the nozzle may be pointed at its end, to allow the nozzle to easily pierce the pod. By piercing the pod, an inlet is created which allows water to flow into and internal cavity of the pod (e.g. through the nozzle).

In some embodiments, such as embodiments in which a hard-shell container is used, the brewer may also pierce the pod a second time to create a hole for discharge of a brewed beverage. That is, an outlet is created through which the brewed beverage may exit the pod (e.g. into a cup).

As will be described in greater detail below, a single-serve container is provided which includes cannabis so that the brewed beverage includes cannabinoids. Accordingly, in some embodiments, a single-serve container is described which delivers cannabinoids using a Keurig™ coffee brewer, a Tassimo™ coffee brewer, or a single-serve brewer of another type.

### Example Container

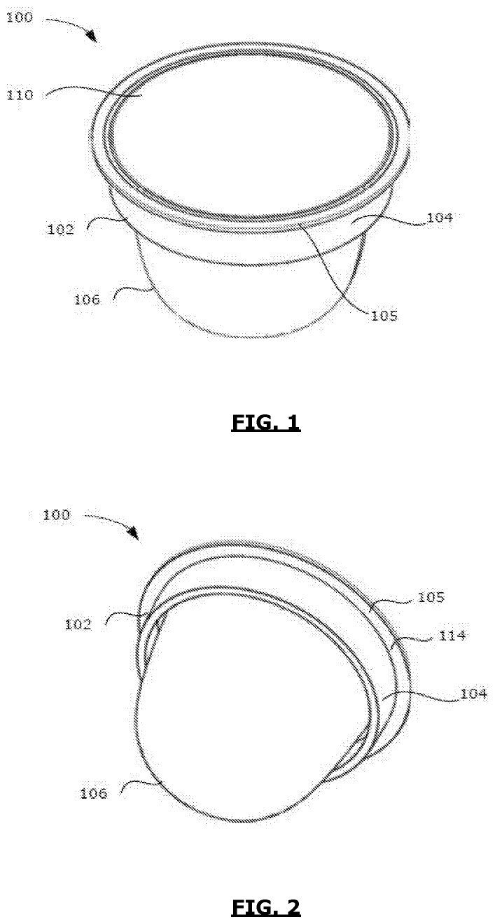

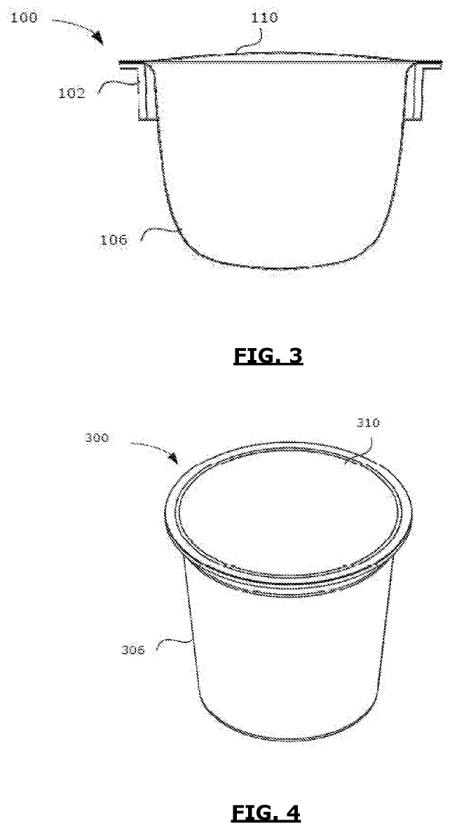

Referring first to FIGS. 1 to 3, an example single-serve container 100 is illustrated. FIG. 1 illustrates the container using a top perspective view, FIG. 2 illustrates the container using a bottom perspective view, and FIG. 3 illustrates the container using a cross sectional view.

In the example illustrated, the single-serve container 100 is constructed of three principal components—a body portion 106, a support ring 102, and a membrane 110. The body portion 106 acts as an exterior shell for the single-serve container and also acts as a filter. This style of container may be referred to as a soft-shell container.

The support ring 102 is constructed of a rigid material, such as a rigid plastic or bioplastic, and functions to maintain the shape of the container 100. The support ring 102 also acts as a support for the container 100 when the container is inserted within a brewing machine of the type described above. More particularly, the brewing machine includes a brewing chamber with a cartridge seat (which may also be referred to as a cartridge holder) which receives the container 100. The support ring 102 contacts the cartridge seat of the brewing machine and holds the container 100 in position for a brew cycle. The diameter of the support ring 102 is sized based on the cartridge seat.

The support ring 102 includes a vertical wall portion 104 and a horizontal portion 105 connected to the vertical wall portion 104. The horizontal portion 105 is the portion that is in the same plane as a top opening of the body portion 106. The horizontal portion 105 provides an upper flat surface which is configured for receiving a membrane 110, which seals the container 100, and a lower flat surface 114 which contacts the cartridge seat of the brewing machine.

The vertical wall portion 104 provides additional rigidity to the support ring and to the container generally. The vertical wall portion 104 may be generally cylindrical in some embodiments. In some such embodiments, the support ring 102 has an L-shaped cross section. However, the vertical wall portion 104 may not be cylindrical in all embodiments and may, for example, be a sloped surface which forms an angle with an axis extending along the center of the support ring 102. For example, the vertical wall portion 104 may form the shape of a truncated cone having two open ends. The vertical wall can take on different shapes and configurations without significantly affecting the functionality of the pod.

The support ring 102 connects with the body portion 106 of the container 100. For example, during construction of the container 100, the support ring 102 may be heat-sealed to the body portion 106. By way of further example, the support ring 102 may be connected to the body portion 106 with a welding process such as ultrasonic bonding or heat bonding. The body portion 106 is generally shaped as an open cup or pocket, or pail.

The body portion 106 is constructed of a filtering material, such as a mesh. The filtering material is permeable to allow a liquid beverage to pass through the body portion 106. However, the filtering material is fine enough to prevent any non-soluble solid contents of a particular size (such as tea, cannabis or coffee grinds) in the container 100 from passing through the body portion 106 during brewing.

In at least some embodiments, the body portion 106 is thermoformed from a flat textile or film. The body portion 106 may, for example, be thermoformed using one or more techniques described in U.S. publication number 20120269933 A1, filed Oct. 19, 2010 by Gino Rapparini, the contents of which are hereby incorporated by reference.

The body portion 106 is generally thermoformed into the shape of a cup or a pail, or pocket, to form an interior space for receiving a powdered cannabis extract (such as microencapsulated cannabis extract produced according to a method described below, which may also be referred to as freeze-dried cannabis extract) and, in at least some embodiments, a flavoring agent such as tea, coffee or chocolate powder mix.

The body portion 106 may also include a rim portion at the open end, which is flat and which sits on the horizontal portion 105 of the support ring 102. The rim portion may be heat sealed to the support ring 102 or attached to the support ring using another technique such as ultrasonic bonding or heat bonding

A membrane 110 seals the container 100. More particularly, the membrane 110 is attached at the open end of the body portion 106 to seal the body portion 106 and provide a cover to the body portion 106. The membrane 110 and the body portion 106 collectively define a cavity which stores the powdered cannabis extract and, in some embodiments, one or more flavoring agents.

The membrane 110 may be a barrier film membrane, which is substantially impermeable to liquids and/or gases. The membrane 110 is thin and rigid, when applied to the container 100, to allow a needle of the brewer to pierce the membrane and extend into the cavity defined by the membrane 110 and the body portion 106 to expel water into the cavity.

In at least some embodiments, the height of the container 100 (i.e. the distance between the membrane 110 and the bottom of the body portion 106) is in the range of 10 to 30 mm and the volume of the cavity is in the range of 15 to 40 mL. In at least some embodiments, the height of the container 100 is in the range of 15 to 35 mm.

The membrane 110 may be attached to other portions of the container 100 after the contents of the pod have been placed into the interior space defined by the body portion 106. The membrane 110 may be attached by heat sealing or ultrasonic welding. For example, in at least some embodiments, the membrane may be attached to a body portion of the container.

The single-serve container illustrated in FIGS. 1 to 3 requires the container to be pierced by only a single needle at a single side. More particularly, when used in a brewing machine, the container 100 is pierced at its top (i.e. through the membrane 110) using a needle. During brewing, the needle injects water into the container 100, which passes through the permeable body portion 106, flows through one or more exit ports in a brewing chamber of the brewing machine (the brewing chamber is the portion of the brewing machine that receives the container 100) and drips into a pot, cup, or other receptacle. This needle may be referred to as a nozzle. In this embodiment, because the pod is defined by a permeable filter, which acts as a housing for the pod, there is no need to puncture the bottom of the pod in order to extract the brewed liquid.

The single-serve container 100 may be configured to be used in available brewing machines, such as a Keurig™ brewing machine.

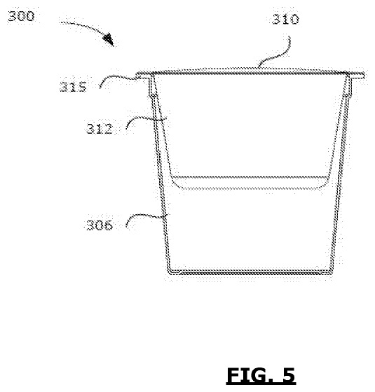

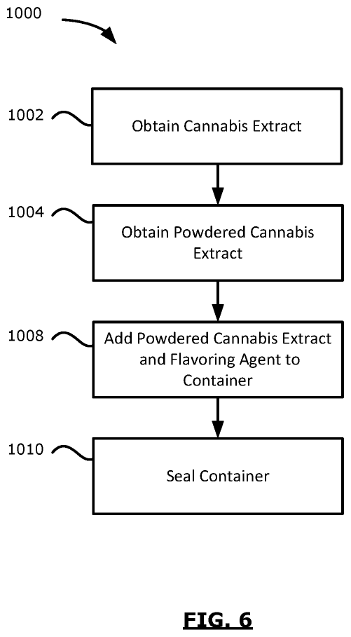

The single serve container may, in other embodiments, take other forms. By way of example, in some embodiments, the single serve container 100 may be a hard shell container. An example hard shell single-serve container 300 is illustrated in FIGS. 4 and 5.

The hard shell single-serve container 300 is constructed of three principal components—a body portion 306, a membrane 310, and a filter 312 (illustrated in FIG. 5).

The body portion 306 is a constructed of a non-permeable (to water or oxygen) material. The body portion 306 is, in at least some embodiments, piercable by an outlet needle associated with a brewing machine. Piercing the body portion 306 creates an outlet which allows a brewed beverage to exit the container 300 during brewing.

In the example illustrated, the body portion 306 has a truncated conical shape. More specifically, the body portion 306 is shaped like a bucket or pail, having a closed bottom end and an open top end. The body portion 306 may have other shapes in other embodiments. For example, in some embodiments, the body portion 306 may be cylindrical.

The body portion 306 performs many of the same functions as the support ring 102 of the embodiment of FIG. 1. For example, the body portion 306 generally maintains the shape of the container 300 and acts as a support for the container 300 when the container 300 is inserted within a brewing machine of the type described above. That is, the container 300 may be placed in a cartridge seat. The body portion 306 may include a ring 315 portion which contacts the cartridge seat of the brewing machine and holds the container 300 in place.

In the embodiment illustrated, the body portion 306 acts as a housing but not a filter. Instead, a filter 312 (illustrated in FIG. 5) is disposed within an interior space defined by the body portion 306 (i.e. the cavity between the membrane 310 and the body portion 306). As can be seen from the sectional view of FIG. 5, the filter 312 may also have a truncated conical shape, having a top open end and a bottom closed end. In other embodiments, the filter 312 may take other shapes, including a cone.

The filter 312, in the example illustrated, is positioned within a cavity that is defined by the body portion 306 and the membrane 310. The filter 312 is constructed of a permeable material such as a mesh or filter paper. In at least some embodiments, the filter may be a nonwoven fabric. Filters 312 may be formed from natural fiber-based material or from a polymer-based material, for example. The filter may, for example, be folded from sheet material or may be stretched from a sheet material. Stretch forming can include thermoforming using heat. The filter 312 is permeable to allow a liquid beverage to pass through the filter, but it is fine enough to prevent any solid contents (such as tea, cannabis or coffee grinds) in the container 300 from passing through the filter 312 during brewing.

In the embodiment illustrated, the top end of the filter 312 is attached to the top end of the body portion 306. This may be done using any of the techniques that are described above for attaching the body portion 106 of the soft-shell container 100 to the support ring 102.

The height of the filter 312 is less than the height of the body portion 306 such that, when the filter 312 is attached to the body portion 306, there is a gap between the filter 312 and the bottom of the body portion 306. This gap allows a drainage needle to pierce the body portion 306 without penetrating the filter 312.

The container 300 is sealed with a membrane 310, which may be of the type described above with reference to the soft-shell container 100. When the membrane 310 is attached to the body portion 306, a first cavity is defined by the membrane 310 and the body portion 306. A second cavity, which is defined by the membrane 310 and the filter 312, is located within the first cavity. The second cavity stores powdered cannabis extract, and, in at least some embodiments, one or more flavoring agents.

The membrane 310 and the body portion 306 may be air-tight. That is, the container 300 may be air-tight. In at least some embodiments, the first cavity defined by the membrane 310 and the body portion 306 may be nitrogen flushed when the membrane 310 is applied to expel oxygen from the cavity and prevent the degradation of the powdered cannabis extract and/or flavouring agent (e.g. Coffee).

Accordingly, the single-serve container may take a variety of forms. Generally, the single-serve container includes a membrane and a body portion. The body portion may be permeable and may be used as a filter in some embodiments (as in the case of the embodiment of FIGS. 1 to 3), or there may be a secondary filter (as with FIGS. 4 and 5).

In some embodiments, the single-serve container is a K-Cup™ container. K-cup containers are described in greater detail in U.S. Pat. No. 5,840,189, the contents of which are hereby incorporated by reference.

In some embodiments, the single serve container may be a T Disc™ style container which is configured for use with a Tassimo™ brewer. T disc style containers are described in U.S. Pat. No. 7,231,869 which was filed on Jan. 23, 2004 and which is incorporated herein by reference.

In some embodiments, the single serve container may be a Nespresso™ container or Nespresso™ compatible container. In some embodiments, the single serve container may be a Nescafe™ Dolce Gusto™ style container.

The containers may take other forms apart from those listed above but the general mode of action is similar. The single serve pod serves to hold and protect the cannabis and other ingredients internally, where the hot water is introduced to extract the actives and then ejected through the brewer head.

### Preparation of Powdered Cannabis Extract

Referring now to FIG. 6, a method 1000 involving powdered cannabis extract is illustrated. The method 1000 may, in some embodiments, use lyophilisation to produce a cannabis powder. More particularly, a freeze-dried cannabis product may be provided. In other embodiments, non-lyophilisation techniques may be used to produce a powdered cannabis extract.

First, at 1002, a concentrated cannabis extract is obtained. The concentrated cannabis extract is manufactured from cannabis. The concentrated cannabis extract may also be referred to as a cannabis oil or resin. The cannabis resin may or may not be diluted to a desired concentration of specific cannabinoids. The cannabis used to manufacture the cannabis extract may be of any strain including pure or hybrid varieties such as Cannabis sativa or Cannabis indica. The cannabis may be harvested before performance of the method 600. The cannabis that is used in the method 600 may include any of the bud, leaves, or fines portions of a cannabis plant, or a combination thereof. In at least some embodiments, the whole flower may be used in the steps described below to produce a whole flower extract.

In at least some embodiments, during 1002, the cannabis may be decarbaroxylated. More particularly, the cannabinoids in the cannabis may be decarboxylated. This process of decarboxylating the cannabinoids in the cannabis is referred to herein as decarboxylating the cannabis. Decarboxylation is the process of removing a carbon dioxide group from a molecule. Decarboxylation converts inactive cannabinoids, such as delta-9-tetrahydrocannabinolic acid (THCA) to active cannabinoids such as delta-9-tetrahydrocannabinol (THC).

The decarboxylation of the cannabis occurs by heating the cannabis. For example, in some embodiments, the decarboxylation is performed by heating the harvested cannabis in an oven (e.g., by baking). The oven may be at a temperature of between 120 degrees Celsius and 140 degrees Celsius and the cannabis may be heated for a time period in the range of 30 to 90 minutes. It will be appreciated that the temperature of the oven and the bake time have an inverse relationship. For example, if the oven is at 120 degrees Celsius, then the bake time may be 60 minutes but if the oven is at 140 degrees Celsius, then the bake time may be only 30 minutes. The bake time is sufficiently long to permit decarboxylation, but short enough that the THC does not appreciably convert to cannabinol (CBN).

In an embodiment, the temperature of the oven is selected to be below the boiling point of the cannabinoids, flavonoids, and terpenoids found in cannabis. Flavonoids are a class of plant pigments. Terpenoids and Flavonoids are, in part, responsible for the look, taste and odor of a particular strain of cannabis. Terpenoids, which are structurally related to terpenes, are naturally occurring in a wide range of plants. In part, they contribute to what gives the plants their unique aromatic quality. Beta-sitosterol is a flavonoid which has a relatively low boiling point of 134 degrees Celsius (as compared with the boiling points of the other flavonoids, cannabinoids and terpenes commonly found in cannabis). Thus, in at least some embodiments, the decarboxylation is performed at a temperature that is less than the boiling point of 134 degrees Celsius. For example, in at least some embodiments, the temperature may be 130 degrees or less.

The oven used in the heating may be a continuous process oven, such as a conveyor oven. A conveyor oven is an oven that is equipped with a conveyor which slowly moves the cannabis through a heating chamber at a predetermined speed until it reaches a position where it is expelled from the heating chamber. For example, cannabis may be added to the conveyor at an upstream end of the conveyor, which may be located outside the heating chamber. The cannabis is then drawn into the heating chamber due to movement of the conveyor and is slowly moved across the length of the heating chamber, where it eventually expelled from the heating chamber at a downstream end of the conveyor.

THC degrades in the presence of oxygen. Accordingly, in some embodiments, the decarboxylation of the cannabis may occur under hypoxic or anoxic conditions to prevent exposure of the cannabis to oxygen. That is, the oven used to decarboxylate may be air-tight and may contain little oxygen so as to prevent the degradation of the cannabis.

Furthermore, since THC degrades in the presence of light, in at least some embodiments, the decarboxylation is performed in a light-free or low-light environment. For example, in some embodiments, the oven is configured to prevent light penetration such that the cannabis is shielded from light during the baking. For example, the oven may be constructed of an opaque housing which prevents penetration of light and the oven may not include any windows.

Other methods of heating the cannabis to decarboxylate the cannabis may be used in other embodiments (i.e. apart from the use of an oven).

At 1002, the decarboxylated cannabis may be pulverized, or otherwise broken apart into small particles. The pulverizing may be performed by impacting, beating, crushing, rolling, grinding or otherwise applying a force to the cannabis to break it apart. The pulverizing may be performed using a pulverizing machine. That is, the cannabis is inserted into the pulverizing machine and the pulverizing machine then impacts the cannabis to break it apart. For example, the pulverizing machine may be of a type commonly used in the food or drug industries.

In some embodiments, the pulverizing may occur under hypoxic or anoxic conditions to prevent exposure of the cannabis to oxygen. That is, the pulverizing machine may be air tight and may provide a low oxygen environment. Further, in some embodiments, the pulverizing may occur under low-light or no-light conditions, to prevent exposure of the cannabis to light. That is, the pulverizing machine may have a light-free pulverizing chamber or region to prevent light from reaching the cannabis while in the pulverizing machine.

In other embodiments, the cannabis may not be protected from oxygen or light during the pulverizing stage. In some such embodiments, the cannabis may be exposed to typical ambient levels of oxygen or light and the pulverizing step may be completed rapidly to ensure that the cannabinoids are not degraded during pulverization. In some embodiments, the pulverizing may be completed within five minutes or less.

The pulverized cannabis may be filtered to ensure a desired and uniform particle size. For example, in some embodiments, the pulverized cannabis may be passed through one or more sieves. The sieves may be used to remove pulverized cannabis particles that are either too big or too small. As noted above, the maximum size for the particles may be set to ensure sufficient surface area of the cannabis.

At 1002, the cannabis (which may be pulverized and/or decarboxylated) is added to a cannabinoid solvent. The cannabinoid solvent may be, for example, an organic solvent, an alcohol (such as ethanol) or an oil. The oil may be, for example, coconut oil (which may or may not be liquid coconut oil in the form of medium chain triglycerides), olive oil, canola oil, or another type of oil. Oils with different fatty acid componsisions may be used to increase THC absorption from the plant. For example, olive and coconut oils have a higher fat content than canola oil.

The cannabis may be washed with the cannabinoid solvent to extract cannabinoids from the cannabis plant into the cannabinoid solvent. This washing may occur repeatedly and the cannabis may be agitated during the washing to enhance extraction. For example, the solvent-cannabis mixture may be mixed, shaken, stirred or otherwise agitated. In some embodiments, the cannabis may be pulverized in the mixture to further enhance extraction.

In some embodiments, after the washing, the cannabis (i.e. the plant material) is filtered from the mixture using a filter.

The mixture may then be heated to boil off the solvent. The heating of the mixture may additionally be used to decarboxylate.

Other methods of making cannabis extracts may be used in other embodiments. For example, in some embodiments, the cannabis extract may be made from cannabis resin glands instead of herbal cannabis and the cannabis extract may, in such embodiments, be referred to as hash oil.

By way of further example, in some embodiments, the cannabis oil may be a carbon dioxide (“CO2”) cannabis extract, which may be referred to as CO2 extract. The CO2 extract may be produced by supercritical or subcritical fluid extraction. More specifically, CO2 may be compressed at high pressures to become a supercritical or subcritical fluid which can then be used to strip the essential oils of the cannabis plant.

In some embodiments, the cannabis extract may be formulated with a cocktail of antioxidants. For example, in some embodiments, ascorbic acid, lecithin and topherol (collectively “ALT”) may be provided in the cannabis oil. In some embodiments, the ALT may be approximately 0.5 to 0.65% by weight. In an embodiment, the ALT may be approximately 0.58% by weight.

The term “cannabis extract” as used herein may include both cannabis oils (which may be oil containing cannabis resin or oil produced by soaking cannabis flowers in oil) and cannabis resins (which are extracted directly from the cannabis plant). At 1004, the cannabis extract, which is in liquid form, is converted into powdered form. That is, at operation 1004 the cannabis extract is converted into a powered cannabis extract. In at least some embodiments, at operation 1004 the cannabis is microencapsulated by freeze drying, which may also be referred to herein as lyophilization. That is, small cannabis extract droplets are transformed into a solid by surrounding the small droplets of extract in a dry matrix of proteins and/or carbohydrates. For example, the cannabis extract may be microencapsulated with sodium caseinate. For example, in an embodiments, sodium caseinate is added to water and is dissolved in the water and the extract is added to the water-sodium caseinate mixture. In one embodiment, the mixture may include approximately ten (10) percent extract by weight, approximately ten (10) percent sodium caseinate by weight and approximately eighty (80) percent water by weight. The amount of these ingredients may vary in other embodiments. For example, in an embodiment, the mixture include five (5) to twenty (20) percent extract by weight.

In another embodiment, the mixture may include maltodextrin. For example, in an embodiment, the sodium caseinate is dissolved in the water and maltodextrin is then added to the mixture. After the maltodextrin is added, the extract may be added. In one embodiment, the mixture may include approximately ten (10) percent extract by weight, approximately ten (10) percent sodium caseinate by weight, approximately ten (10) percent maltodextrin by weight and approximately seventy (70) percent water by weight. The amount of these ingredients may vary in other embodiments. For example, in an embodiment, the mixture include five (5) to twenty (20) percent extract by weight.

In some embodiments, sodium alginate may be used instead of or in addition to sodium caseinate. Other hydrocolloids may be used for microencapsulation in other embodiments including, for example, gellan gum, guar gum, gum Arabic, k-carrageenan, konjac glucomannan, mesquite gum, pectin, or xanthan gum.

After the ingredients have been added together, the mixture may be homogenized to evenly disperse the ingredients. For example, the mixture may be homogenized using a homogenizer.

The homogenized mixture (which may be referred to as a solution in some embodiments) is then freeze dried. More specifically, the solution is frozen at a desired freezing rate and is dried under desired drying conditions. The freeze drying may be performed using a freeze drying machine.

The powdered cannabis extract (i.e., the product of operation 1004 which may be microencapsulated by freeze drying as described above or converted into a powder using another technique), which is in solid form, is added to a single-serve container at 1008. The container may be of any of the types described above including, for example, the type of container 100 described above with reference to FIGS. 1 to 3, the container 300 described with reference to FIGS. 4 and 5, a K-cup, a T Disc™ style container, a Nespresso™ container or Nespresso™ compatible container, a Nescafe™ Dolce Gusto™ style container, or a single-serve container of another type which has a form factor that is configured for receipt within a single-serve beverage dispenser including a single-serve brewing machine such as a single-serve coffee maker.

The powdered cannabis extract is added to an interior space of the container (such as a single serve container 100, 300 of the type described above) at 1008. In some embodiments, the powdered cannabis extract may be added to the container using a hopper. The hopper feeds a filling head which dispenses the powdered cannabis extract into the container. For example, the filling head may be associated with a screw mechanism that pushes a specific amount out of the filling head by way of controlled rotation of the screw. In this way, the amount of powdered cannabis extract added to the container may be strictly controlled.

The amount of powdered cannabis extract added to the container is predetermined and will depend on the desired dosage level for the container.

The addition of the powdered cannabis extract is performed with strict tolerance controls regarding the quantity of powdered cannabis extract added so that each container receives the desired quantity of powdered cannabis extract within an accepted tolerance level (e.g. 2% in some embodiments). As a result, the THC and CBD (or THCA and CBDA) levels in each beverage produced using the containers at a given dosage level are highly consistent. Such consistency is not found in smoking or vaporization, which are currently the most common methods of using cannabis.

Where a container 100 of the type described above with reference to FIGS. 1 to 3 is used, the powdered cannabis extract may be added to the cavity defined by the membrane 110 and the body portion 106. In other embodiments, a container 300 of the type described above with reference to FIGS. 3 and 4 is used. In some such embodiments, the powdered cannabis extract is added to the cavity defined by the filter 312 and the membrane 310. In other embodiments in which a container 300 of the type described above with reference to FIGS. 4 and 5 is used, the powdered cannabis extract is added between a cavity that exists between the filter 312 and the body portion 306. That is, the powdered cannabis extract may be provided in the gap between the filter 312 and the bottom of the body portion 306 (FIG. 4). This gap allows a drainage needle to pierce the body portion 306 without penetrating the filter 312. By placing the powdered cannabis extract in this location, when the container 300 is pierced with the needle, the powdered cannabis extract may fall into a cup through the hole created by the needle.

Other ingredients may also be added to the container during step 1008. For example, in some embodiments, a flavoring agent is added. The flavoring agent may be used to enhance the flavor of the beverage created when the container is used in a single-serve beverage machine. In at least some embodiments, the flavoring agent may provide a beverage that a user might ordinarily consume, even if they were not consuming a cannabis-based beverage. That is, the flavoring agent may be a common beverage with a flavor for any common beverage. For example, in some embodiments, the flavoring agent is coffee and, more particularly, is roasted and ground coffee. The ground coffee may be complemented with additional flavoring to enhance the coffee flavor such as, for example, irish cream flavoring, vanilla flavoring, hazelnut flavoring, etc.

In at least some embodiments, the ground coffee has a particle size that ranges between 291 and 570 microns. In at least some embodiments, some grinds may have a larger particle size. For example, in some embodiments, at least 90% of grinds are 570 microns or less.

In some embodiments, the flavoring agent may be tea including, for example, black tea, green tea, white tea, or other teas. The tea may include cured leaves of the tea plant, Camellia sinesis and/or may include other ingredients such as fruits or herbs. For example, in some embodiments, the tea may be one of rosehip tea, chamomile tea, or rooibos tea.

In at least some embodiments, the tea has a density of between 95-200 grams per 500 ml. In some embodiments, the maximum moisture of the tea is 12% or less. In some embodiments, the tea has a fanning in the range of 0.2-2.5 mm for the bulk of the sample (e.g. at least 80% of the tea is within this range).

In some embodiments, the flavoring agent may be chocolate which may be provided in powder form. In at least some embodiments, cocoa powder may be used. In some embodiments, the chocolate powder may be made by agglomerating cocoa powder, sugar, milk powder or a non-dairy creamer and other flavours and/or artificial sweeteners. The ingredients may be agglomerated to a specific size and density in order to prevent filtering through the filter (which may be a filter or a permeable body portion of a container), to facilitate dissolution during brewing and to ensure sufficient quantities can be delivered to maximize the sensory impact of the powder.

In at least some embodiments, the chocolate powder has a particle size that ranges between 291 and 570 microns. In at least some embodiments, some particles may have a larger particle size. For example, in some embodiments, at least 90% of particles are 570 microns or less.

In some embodiments, the flavoring agent may be a syrup or concentrate such as, for example, a soda-flavored or fruit-flavored syrup or concentrate. By way of example, the syrup may be cola-flavored. By way of further example, in some embodiments, the flavoring agent may be cappuccino or latte. Liquid flavoring agents may not work with all containers described herein or may benefit from modifications to at least some the containers described herein.

The quantity of flavoring agent used will depend on the nature of the flavoring agent used. For example, 6 to 12 grams of coffee may be used. In other embodiments, 9 to 20 grams of coffee may be used. By way of further example, in some embodiments, 3 to 7 grams of tea may be used. In yet other embodiments, 6 to 14 grams may be used. By way of further example, in some embodiments, 6 to 10 grams of chocolate powder may be used. In some embodiments, 8 to 20 grams of chocolate powder may be used.

The flavoring agent may, in some embodiments, be a combination of two or more of the flavoring agents described above. In some embodiments, the flavoring agent may be a matcha latte powder

The flavoring agent may, in some embodiments, be mixed with the powdered cannabis extract to create a mixture and then the mixture is added to the container (e.g. the flavoring agent and powdered cannabis extract may be added via a common hopper or filler).

In some embodiments, the flavoring agent may be added separately from the powdered cannabis extract. For example, in some embodiments, the powdered cannabis extract is added using a first hopper and the flavoring agent is added using a second hopper.

In some embodiments, the flavoring agent and the powdered cannabis extract are added to a common compartment within the container. That is, the flavoring agent and the cannabis may contact one another within the container since there is no physical barrier separating the flavoring agent and the cannabis within the container. In other embodiments, the container may be constructed with a physical barrier which separates the interior space of the container into multiple chambers. One of those chambers may store the powdered cannabis extract while the other may store the flavoring agent.

Furthermore, in some embodiments, an emulsifier is added to the container. The emulsifier may be lecithin, for example. When a beverage is created using the single-serve container, the emulsifier allows the extract droplets containing the cannabinoids to disperse within the beverage to create a more homogeneous and palatable beverage.

In some embodiments, two or more ingredients may be premixed prior to operation 1008 of the method 1000. For example, the powdered cannabis extract may be pre-mixed with the flavoring agent and the pre-mixture could then be added to the single-serve container at operation 1008.

At 1010, the single-serve container 100, 300 is sealed. For example, a membrane 110, 310 may be applied to the container 100, 300. The membrane acts as a lid to the container, closing the interior space of the container. The membrane may be configured to be pierced by a nozzle associated with the beverage machine. The membrane is, in some embodiments, applied using heat. In some embodiments, the membrane is applied using induction sealing or ultrasonics.

In some embodiments, such as the embodiment of FIGS. 4 and 5, the membrane provides an air-tight seal for the container 300 so that air cannot enter or exit the container's interior space. In at least some such embodiments, to prevent degradation of the cannabinoids, the oxygen within the interior space is displaced. That is, the oxygen in the vicinity of the decarboxylated cannabis may be displaced. This displacement may be performed, for example, by nitrogen flushing the container. That is, the interior space of the container is nitrogen flushed during the application of the membrane so that, once sealed, the container contains only minute amounts of oxygen (e.g., less than 1%).

In other embodiments, the membrane may not provide an air-tight seal. For example, where the container is constructed in the manner illustrated in FIGS. 1 to 3 so that the container's body portion is permeable, the membrane does not provide an air-tight seal. Instead, a non-permeable overwrap may be applied to the single-serve container and the oxygen may be displaced within the overwrap. For example, the overwrap may be vacuum sealed and/or nitrogen flushed. Once sealed, the container 100, 300 (and the cannabis inside the container) is only exposed to trace amounts of oxygen (e.g., less than 1%).

In at least some embodiments, the overwrap may be constructed of a thin laminate material with good oxygen & moisture barrier properties which may be formed and sealed so that it resembles a potato-chip bag or small pillow pack. To prevent degradation of the cannabinoids by light, the overwrap may be opaque.

In at least some embodiments, the method 1000 or a portion thereof may be an automated process.

A container of a type described herein (and containing ingredients of the type described herein) may, in at least some embodiments, be used according to one or more of the following methods to brew a beverage. First, the container is inserted into a single-serve brewing machine. For example, the container may be placed on a cartridge seat of the brewing machine. The membrane of the container is pierced by the brewing machine, typically when the brewing machine is closed. More particularly, a needle pierces the membrane and enters the interior space defined by the container's body portion and membrane. Depending on the container's configuration (e.g., whether it is a hard shell container or a soft shell container), the bottom of the container may also be pierced to create an exit port for a brewed beverage. An instruction is then input into the brewing machine to instruct the brewing machine to initiate a brew cycle. This instruction may, for example, be input to the brewing machine using a button or other input mechanism associated with the machine. The instruction is received at the brewing machine in the form of a signal (e.g. an electrical signal).

After the needle pierces the brewing machine and in response to receiving the instruction to initiate the brewing cycle, water (which may be heated) is injected into the container from the needle. This water mixes with the powdered cannabis oil and creates a beverage which includes active cannabinoids. The brewed beverage exits the container either through the permeable body portion (i.e., if the container is a soft shell container) or through the exit port created by piercing the container (i.e., if the container is a hard shell container).

After the brew cycle has completed, the brewed beverage may then be consumed.

The method 1000 described above may, in an embodiment, be modified to use a different technique to turn the cannabis oil into a powder. For example, non-lyophilisation methods may be used to convert the cannabis extract into powder at operation 1004. That is, the powdered cannabis extract may be prepared without freeze drying. For example, a cannabis extract which is an oil may be converted into a powder by mixing with maltodextrin (which may be from any source such as tapioca, corn, wheat, etc.). Gums, pectins, proteins and other similar agents may also be added to the mixture.

By way of example, at operation 1004 of the method 1000, the cannabis extract obtained at operation 1002 may be made into a powder by mixing the cannabis extract with absorbent carriers such as silica and maltodextrin. The silica may be a high porosity spherical silica such as fumed silica or various silica salts. The silica may have a small particle size (for example, the average silica particle size distribution may be about 1.01 to 0.05 microns). Maltodextrin, or another suitable carrier that will dilute and absorb high viscosity liquids, may also be mixed with the cannabis extract. The combination of silica and maltodextrin may allow for better absorption of high viscosity fluids than if only maltodextrin were used. Other absorbent materials that may be used include carbohydrates, proteinaceous materials such as sodium casienate, soy isolate, or whey protein, and fibers such as pectin, guar gum, or carboxymethylcellulose.

It will be appreciated that the techniques described herein may be used to create a packaged beverage product which may be delivered in another form, apart from the single serve container described above. For example, in some embodiments, powdered cannabis extract may be packaged with other ingredients of the types described above (e.g. the flavoring agent) for creating a steeped or brewed beverage. For example, in some embodiments, such contents may be packaged in a tea bag. In some embodiments, the contents may be sold in a blended form (e.g. the powdered cannabis extract and, in some embodiments, flavoring agent) may be blended and sold. Such contents could then be added to a tea infuser, filter brewing machine, percolator, or French press, for example, to create a beverage. For example, when a tea infuser is used the tea infuser will be placed in hot or cold water.

Further, in some embodiments, the decarboxylation may occur after the cannabis extract is obtained. For example, the cannabis extract may be obtained from cannabis that has not yet been decarboxylated and the cannabis extract may then be heated to decarboxylate the cannabis extract.

Techniques described herein may be used to convert liquid cannabis extract to a powdered cannabis extract, which is a solid. The powdered cannabis extract may be packaged separately (e.g., apart from the flavoring agent), or may be included in other products apart from the single-serve containers described herein.

The various embodiments presented above are merely examples. Variations of the innovations described herein will be apparent to persons of ordinary skill in the art, such variations being within the intended scope of the present application. In particular, features from one or more of the above-described example embodiments may be selected to create alternative example embodiments including a sub-combination of features which may not be explicitly described above. In addition, features from one or more of the above-described example embodiments may be selected and combined to create alternative example embodiments including a combination of features which may not be explicitly described above. Features suitable for such combinations and sub-combinations would be readily apparent to persons skilled in the art upon review of the present application as a whole. The subject matter described herein and in the recited claims intends to cover and embrace all suitable changes in technology.

What is claimed is:

1. A method of preparing a single-serve container configured for receipt in a single-serve brewing machine, the method comprising:
obtaining cannabis extract;
processing the cannabis extract to obtain powdered cannabis extract and adding the powdered cannabis extract to the single-serve container;
adding a flavoring agent to the single serve container; and
sealing the single-serve container.

2. The method of claim 1, wherein obtaining the cannabis extract comprises decarboxylating cannabis.

3. The method of claim 2, wherein decarboxylating the cannabis comprises:
heating the cannabis in an oven.

4. The method of claim 3, wherein heating comprises baking at a temperature of between 120 and 140 degrees Celsius for a time period in the range of 30 to 90 minutes.

5. The method of claim 1, wherein the flavoring agent is one of: tea, coffee or chocolate, latte or cappuccino.

6. The method of claim 1, wherein processing the cannabis extract to obtain powdered cannabis extract comprises:
microencapsulating the cannabis extract by freeze drying.

7. The method of claim 6, wherein the cannabis extract is microencapsulated in sodium caseinate.

8. The method of claim 1, wherein processing the cannabis extract comprises mixing the cannabis extract with an absorbent carrier.

9. The method of claim 1, wherein processing the cannabis extract comprises:
mixing sodium caseinate together with water and the cannabis extract to obtain a mixture;
homogenizing the mixture; and
freeze drying the homogenized mixture.

10. The method of claim 9, wherein the mixture comprises approximately 10% sodium caseinate by weight, approximately 10% cannabis extract by weight and approximately 80% water by weight.

11. The method of claim 9, wherein mixing further comprises mixing maltodextrin with the sodium caseinate, water and cannabis extract.

12. The method of claim 11, wherein the mixture comprises approximately 10% sodium caseinate by weight, approximately 10% cannabis extract by weight, approximately 10% maltodextrin by weight and approximately 70% water by weight.

13. (canceled)

14. A method of converting liquid cannabis extract into a solid, the method comprising:
obtaining the liquid cannabis extract; and
processing the cannabis extract to obtain powdered cannabis extract.

15. The method of claim 14, wherein obtaining the cannabis extract comprises decarboxylating cannabis.

16. The method of claim 15, wherein decarboxylating the cannabis comprises:
heating the cannabis in an oven.

17. The method of claim 16, wherein heating comprises baking at a temperature of between 120 and 140 degrees Celsius for a time period in the range of 30 to 90 minutes.

18. The method of claim 14, wherein processing the cannabis extract to obtain powdered cannabis extract comprises:
microencapsulating the cannabis extract by freeze drying.

19. The method of claim 18, wherein the cannabis extract is microencapsulated in sodium caseinate.

20. The method of claim 14, wherein processing the cannabis extract comprises: mixing sodium caseinate together with water and the cannabis extract to obtain a mixture;
homogenizing the mixture; and
freeze drying the homogenized mixture.

21. The method of claim 20, wherein the mixture comprises approximately 10% sodium caseinate by weight, approximately 10% cannabis extract by weight and approximately 80% water by weight.

22. The method of claim 20, wherein mixing further comprises mixing maltodextrin with the sodium caseinate, water and cannabis extract.

23. The method of claim 22, wherein the mixture comprises approximately 10% sodium caseinate by weight, approximately 10% cannabis extract by weight, approximately 10% maltodextrin by weight and approximately 70% water by weight.
